(19) United States
(12) Patent Application Publication
(10) Pub. No.: US 2005/0048023 A1
(43) Pub. Date: Mar. 3, 2005

(54) Composition comprising at least one conductive polymer and at least one fluorescent dye and/or at least one optical brightener, and process for use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,471

(22) Filed: Jul. 16, 2004

Related U.S. Application Data
(60) Provisional application No. 60/492,299, filed on Aug. 5, 2003.

(30) Foreign Application Priority Data
Jul. 16, 2003 (FR) 03 08673

(51) Int. Cl. A61K 7/021; A61K 7/06; A61K 7/11

(52) U.S. Cl. 424/70.17; 424/63

(57) ABSTRACT

The present disclosure relates to a composition comprising, in a cosmetically acceptable medium, at least one fluorescent dye and/or an optical brightener, and at least one conductive polymer. The present disclosure also relates to a process for treating keratin fibers, for instance human keratin fibers, such as the hair using such a composition to give keratin fibers an optical effect.

This application claims benefit of U.S. Provisional Application No. 60/492,299, filed Aug. 5, 2003.

The present disclosure relates to a composition comprising, in a cosmetically acceptable medium, at least one conductive polymer and at least one fluorescent dye and/or at least one optical brightener. The present disclosure also relates to a process for treating keratin fibers using the composition as disclosed herein, and to the use of this composition to give keratin fibers an optical effect.

The present disclosure further relates to the field of treating keratin fibers, for instance human keratin fibers such as the hair.

The present disclosure also relates to dyeing processes.

There are mainly two major types of hair dyeing processes. The first type is semi-permanent dyeing or direct dyeing, which uses dyes capable of giving the hair's natural coloration a more or less pronounced change that withstands shampooing several times. These dyes are known as direct dyes and can be used, for example, in two different ways. The first comprises simply applying the composition containing the direct dye(s) to the keratin fibers. The second comprises applying the composition in the presence of an oxidizing agent, under alkaline pH conditions. Such a process can be referred to as lightening dyeing.

Although improvements have been made in this field, direct dyes can still result in colorations with strength and fastness properties that remain to be improved. The second major type of hair dyeing is permanent dyeing or oxidation dyeing. This is obtained with “oxidation” dye precursors, which are colorless or weakly colored compounds. Once mixed with oxidizing products, at the time of use, these precursors lead to colored and coloring compounds via a process of oxidative condensation.

In the case of oxidation dyeing, the colorations obtained can be generally very fast and strong. The problem is that they require the presence of an oxidizing agent used under alkaline pH conditions. In the long run, such conditions may cause more or less pronounced degradation of the treated fibers, which can, for example, impair their shiny appearance.

It should be noted that this phenomenon of degradation of the fibers may also be observed when a direct dye is used under lightening dyeing conditions.

In order to compensate for such drawbacks, the fibers may be treated with an agent that gives them sheen, for example. Among the usual agents that may be mentioned are lubricating hydrophobic substances, such as organic oils or waxes or silicones. This agent may be present in the dye composition itself, or may be provided in a composition applied after dyeing. It is generally desired to perform a post-treatment rather than to use a dye composition comprising the said agent. The reason for this is that it is not uncommon to observe with these compositions poorer uptake of the dye into the fiber, leading to less intense and less fast colorations.

Despite all this, whatever the option adopted, the sheen effect obtained with these agents usually lacks intensity and often gives the fibers an artificial look. Finally, these compositions may have the drawback of giving the fibers a greasy or tacky feel.

There is thus a need for means for dyeing keratin fibers that would limit the phenomena of impairment following repeated treatment, for instance the tendency of the fibers to become more or less coarse, brittle and dull, which would allow access to colorations that are possibly lighter, without it being necessary to use an oxidizing agent; which would give the fibers an aesthetic optical effect, for instance a sheen effect, without having the drawbacks of the conventional methods.

Accordingly, one aspect of the present disclosure is thus a composition comprising, in a cosmetically acceptable medium:
at least one fluorescent dye and/or an optical brightener, and
at least one conductive polymer.

The present disclosuremoreover relates to a process for dyeing keratin fibers, for instance, human keratin fibers, such as the hair, which comprises:
applying to wet or dry keratin fibers a composition as disclosed herein, which is left on the fibers for a time that is sufficient to obtain a desired coloration effect;
optionally rinsing the fibers,
optionally washing and rinsing the fibers,
drying the fibers or leaving them to dry.

According to another aspect, the process according to the present disclosure comprises:
applying to wet or dry keratin fibers a composition according to the present disclosure and not comprising any oxidation base, coupler or oxidizing agent,
drying the fibers or leaving them to dry.

The composition according to the present disclosure can uniformly give all the keratin fibers an optical effect, for instance, a sheen that can be more intense, more natural and more aesthetic than with the means of the prior art.

Moreover, the presence of the at least one fluorescent dye can also give the keratin fibers a different coloration from the color before the treatment, or even a lighter color, without requiring the presence of an oxidizing agent used under basic pH conditions.

The coloration may also be modified when the at least one conductive polymer present in the composition absorbs in the visible spectrum.

Finally, the keratin fibers treated in accordance with the present disclosure can have a soft, non-greasy feel.

Other characteristics and benefits of the present disclosure will emerge more clearly upon reading the description and the examples that follow.

In the text hereinbelow and unless otherwise indicated, the limits of a range of values are understood as forming part of that range.

For the purposes of the present disclosure, the term “optical effect” covers sheen, color, metallic, goniochromatic and moire effects.

Moreover, it should be noted that the sheen corresponds to the light intensity reflected at an angle α when the lock of hair is illuminated under an angle -α. The angle α conventionally used to measure this specular reflection, i.e., the sheen, is equal to 20°. This provision of sheen may be measured using a glossmeter as described, for example, in ISO standard 2813-1994 from AFNOR (August 1994, amended February 1997).

According to the present disclosure, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

For the purposes of the present disclosure, the expression “conductive polymer absorbing in the visible spectrum” means any conductive polymer having a non-zero absorbance in the wavelength ranging from 400 to 800 nm, even if the absorption maxima of the polymer are outside this range.

The conductive polymers that may be used in the context of the present disclosure are conductive polymers that are soluble or dispersible in the cosmetic medium suitable for use. The polymer is said to be soluble in the medium when it forms an isotropic clear liquid at 25° C. in the medium comprising water or a mixture of water and at least one organic solvent, this being obtained throughout all or part of a concentration of conductive polymer ranging from 0.01% to 50% by weight.

In one embodiment of the present disclosure, for example, the at least one conductive polymer used in the composition as disclosed herein is a conductive polymer that is soluble or dispersible in an aqueous medium, for instance, in water.

The polymer is said to be dispersible in the medium comprising water or mixture of water and at least one organic solvent if, at 0.01% by weight, at 25° C., it forms a stable suspension of fine, generally spherical particles. The mean size of the particles comprising the dispersion is less than 1 μm, for example, ranging from 5 nm to 400 nm, such as from 10 nm to 250 nm. These particle sizes are measured by light scattering.

It is worth noting that these polymers do not require the use of a dispersant.

In another embodiment of the present disclosure, the at least one conductive polymer is in a form that is soluble in the medium of the composition.

In general, the conductive polymers can have, for example, a conductivity ranging from 10⁻⁵ to 5×10⁵ siemens/cm, for instance, from 10⁻³ to 10⁵ siemens/cm, such as from 10⁻¹ to 10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space (d), are applied by simple pressure to the sample to be analyzed. A current (I) is injected via the outer points using the current source, thus creating a variation in potential. The voltage (U) is measured between the two inner points connected to the voltmeter of the current generator.

In this configuration, the conductivity of the sample expressed in siemens (S) per centimetre, or S/cm, is given by the following expression:
σ=(K×I)/(U×e)
wherein:

K is a coefficient depending on the position of the contacts on the surface of the sample. When the points are aligned and equidistant, K is equal to: π/log(2)

I: value of the injected current, expressed in amperes

U: the measured voltage value, expressed in volts

e: thickness of the sample, expressed in cm.

This expression can be used only when the thickness of the material is negligible compared with the distance (d) existing between two points (e/d<0.25). In order to obtain sufficiently small thicknesses and thus to be able to calculate the conductivity of the material, one may perform the measurement on a non-conductive support (for example a glass slide) coated with the material to be analyzed, obtained by evaporation of a dilute solution. In order to improve the homogeneity of the coating to be analyzed, one may use the deposition technique known as spin coating.

According to another embodiment of the present disclosure, the at least one conductive polymer is chosen from polymers comprising at least one repeating unit of the following formulae:
anilines of formula (I): 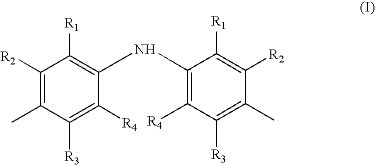
pyrroles of formulae (IIa) and (IIB): 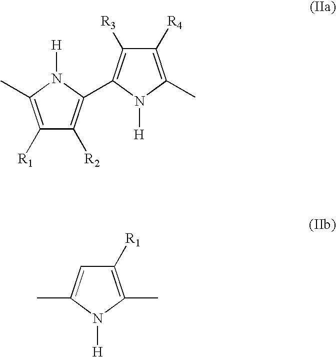
thiophenes and bisthiophenes of formulae (IIIa), (IIIb) and (IIIc): 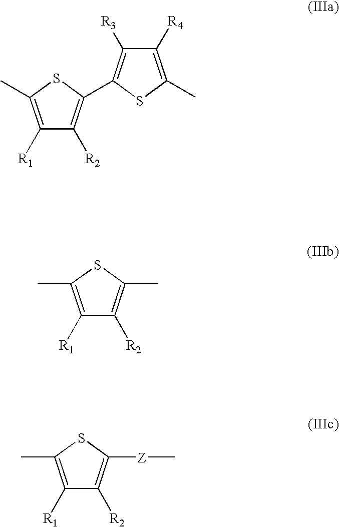
furans of formula (IV): 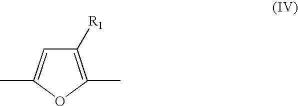
para-phenylene sulfides of structure (V): 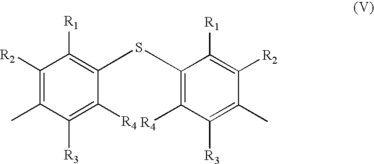
para-phenylenevinylenes of formula (VI): 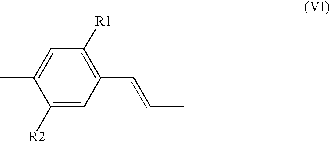
indoles of formula (VII): 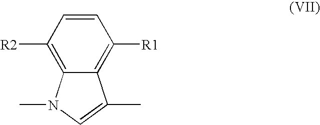
aromatic amides of formulae (VIIIa), (VIIIb), (VIIIc) and (VIIId): 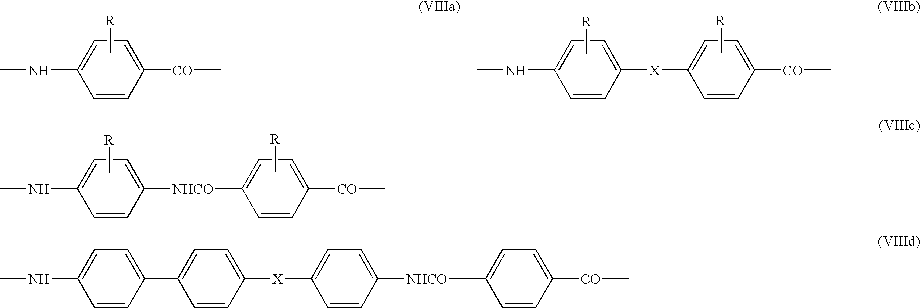
aromatic hydrazides of formulae (IXa), (IXb) and (IXc): 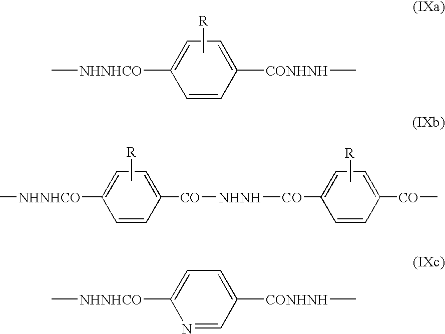
aromatic azomethines of formulae (Xa), (Xb) and (Xc): 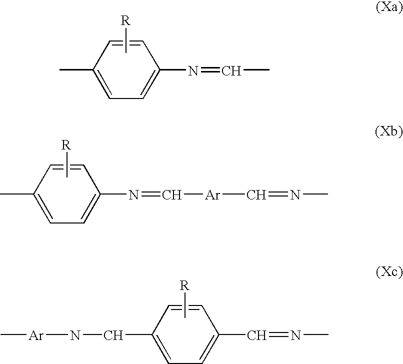
aromatic esters of formulae (XIa), (XIb) and (XIc): 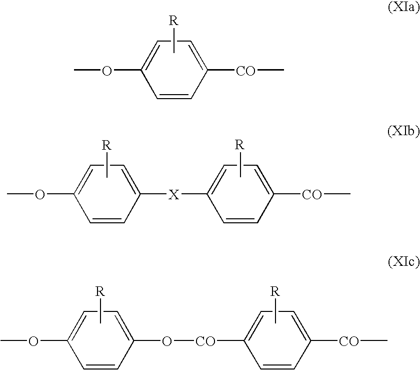
wherein in formulae (I) to (XI):

the radicals R, R1, R2, R3, and R4, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups, and solubilizing groups comprising a spacer group that bonds to the ring;

Ar is a radical comprising a monoaromatic or polyaromatic radical;

X is chosen from oxygen and sulfur atoms, and —NHCO—, —SO₂—, —N═N—, —C(CH₃)₂—, —CH₂—, —CH═CH—, and —CH═N— radicals; and

Z is chosen from —CH═CH— and —C≡C— radicals.

By way of non-limiting example, Ar may be at least one radical chosen from:

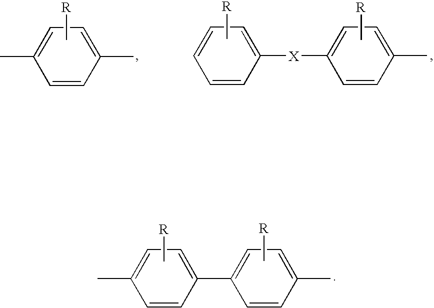

For the purposes of the present disclosure, the term “solubilizing group” means a group that ensures the dissolution of the said molecule in the cosmetic medium, such that the polymer has a conductive nature after drying the composition.

The at least one conductive polymer present in the composition as disclosed herein may comprise at least one repeating unit comprising at least one solubilizing group, and at least one other such units lacking a solubilizing group.

Among the solubilizing groups that may be used, non-limiting mention may be made of those chosen from:
carboxylic (—COOH) and carboxylate (—COO-M⁺) radicals, wherein M is chosen from alkali metals, such as sodium or potassium, alkaline-earth metals, organic amines such as a primary, secondary or tertiary amines, alkanolamines and amino acids,
sulfonic (—SO₃H) and sulfonate (—SO₃⁻M⁺) radicals, wherein M has the same definition as above,
primary, secondary and tertiary amine radicals,
quaternary ammonium radicals such as —NR′₃⁺Z⁻ wherein Z is chosen from Br and Cl atoms, and (C₁-C₄)alkyl-OSO₃ radicals, and wherein R′, which may be identical or different, are chosen from linear and branched C₁ to C₂₀ alkyls, or two R′ radicals may form a heterocycle with the nitrogen,
hydroxyl radicals, and
poly((C₂-C₃)alkylene oxide) radicals.

The carboxylic and sulfonic acid functional groups may optionally be neutralized with a base, such as sodium hydroxide, 2-amino-2-methylpropanol, triethylamine or tributylamine, for example.

The amine radicals may optionally be neutralized with a mineral acid, such as hydrochloric acid, or with an organic acid, such as acetic acid or lactic acid, for example.

In addition, it is possible for the solubilizing radicals to be connected to the ring via a spacer group, for instance a spacer group chosen from —R″—, —OR″—, —OCOR″— and —COOR″— radicals, wherein R″ is chosen from linear and branched C₁-C₂₀ alkyl radicals optionally comprising at least one hetero atom, for instance oxygen.

For example, the radicals R, R1, R2, R3, and R4, which may be identical or different, are chosen from hydrogen atoms, and R′, —OR′, —OCOR′ and —COOR′ radicals, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃ with x being an average number ranging from 0 to 200.

The number n of repeating units in the polymer can range from 5 to 10,000, for instance from 5 to 1000, such as from 10 to 1,000 and from 20 to 700.

For example, at least one radical chosen from R, R1, R2, R3, and R4 of the at least one conductive polymer may be a solubilizing group.

In accordance with one embodiment of the present disclosure, for example, the at least one conductive polymer comprises at least one solubilizing group per repeating unit.

In another embodiment of the present disclosure, the at least one conductive polymer is soluble in the medium of the composition.

The conductive polymers that may be used in the composition as disclosed herein are known to those skilled in the art and are described for example, in the book “Handbook of Organic Conductive Molecules and Polymers”—Wiley 1997—New York, Vol 1, 2, 3, and also in the review Can. J. Chem. Vol 64, 1986.

Polythiophenes and their synthesis are described for instance in the article taken from the review Chem. Mater. 1998, Vol. 10, No. 7, pages 1990-1999, by the authors Rasmussen S. C., Pickens J. C. and Hutchison J. E. “A new, general approach to tuning the properties of functionalized polythiophenes: The oxidative polymerization of monosubstituted bithiophenes”; in the article taken from the review Macromolecules 1998, 31, pages 933-936, by the same authors “Highly conjugated, water-soluble polymers via direct oxidative polymerization of monosubstituted bithiophenes”. In addition to polymerization via chemical or electrochemical oxidation, they may also be obtained by polycondensation (dihalothiophene; catalysis with nickel or palladium complexes); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond in the presence of a Pd-based catalyst—AA-BB or A-B type); via Reike polymerization (organozinc in the presence of a nickel complex); via polymerization of McCulloch type, and so forth.

The conductive polymers that may be used in the composition according to the present disclosure are moreover described in international patent application WO 99/47570.

Among the conductive polymers that are suitable for use as disclosed herein, non-limiting mention may be made, for example, of the polymers of formulae (IIIa), (IIIb) and (IIIc) wherein the solubilizing groups may be chosen from, for instance, carboxylic acid radicals; sulfonic acid radicals; tertiary amine radicals; quaternary ammonium radicals such as —NR′₃⁺Z⁻ wherein Z is chosen from Br and Cl atoms, and (C₁-C₄)alkyl-OSO₃ radicals and wherein R′, which may be identical or different, are chosen from linear and branched C₁ to C₂₀ alkyl radicals, or two R′ radicals form a heterocycle with the nitrogen; the solubilizing groups being optionally connected to the ring via a spacer. The carboxylic and sulfonic acid radicals may optionally be neutralized.

Thus, the polymerization may be performed via chemical or electrochemical oxidation of the corresponding thiophene monomer or else via polycondensation.

By way of non-limiting illustration, the polythiophenes of formulae (IIIa) and (IIIb) may be obtained by polymerization via oxidation (for example with FeCl₃ catalysis); via polycondensation of dihalothiophene catalyzed with nickel or palladium complexes (e.g.: NiCl₂(dppe)₂); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond formed in the presence of a Pd-based catalyst—AA-BB or A-B type); via Reike polymerization (organozinc in the presence of a nickel complex); via polymerization of McCulloch type, and so forth.

The vinylene polythiophenes of formula (IIIc) wherein Z is a —CH═CH— radical, may be obtained for instance, via Gilch polymerization in the presence of a strong base (potassium tert-butoxide) of 2,5-bis(bromoalkylene)thiophene; via polymerization by the Wessling method via the use of a precursor based on sulfonium salts and pyrolysis; and via a Wittig-Horner Wittig reaction.

The ethynylene polythiophenes of formula (IIIc) wherein Z is a —C≡C— may be obtained by Heck-Sonogashira coupling (of the type AA-BB or A-B; formation of a carbon-carbon bond between a terminal acetylenic (or true acetylenic) functional group and a bromo or iodo functional group, catalyzed with a palladium/copper complex (PdCl₂(PPh₃)₃, Cul or Cu(OAc)₂) in the presence of a base such as triethylamine, diisopropyl amine, piperidine, etc.); via metathesis of alkynes in the presence of a molybdenum complex (Mo(CO)₆).

In general, the functionalization of the polythiophenes, i.e., the introduction of the solubilizing or non-solubilizing group(s), is performed on the monomer before it is polymerized. In other cases, the solubilizing group is obtained after working up the polymer. This is the case, for example, for the carboxylic acid functional group, which may be obtained by hydrolysis of the corresponding ester.

For example, the solubilizing groups may be chosen from carboxylic acid radicals; sulfonic acid radicals; tertiary amine radicals; quaternary ammonium radicals such as —NR′₃⁺Z⁻ wherein Z is chosen from Br and Cl atoms, and (C₁-C₄)alkyl-OSO₃ radicals and wherein R′, which may be identical or different, are chosen from linear and branched C₁-C₂₀ alkyl radicals; and the salts thereof wherein the solubilizing groups are optionally connected to the ring via a spacer, such as a spacer chosen from C₁-C₂₀ alkyl radicals. The carboxylic and sulfonic acid radicals may optionally be neutralized.

According to another embodiment of the present disclosure, the at least one conductive polymer is chosen from polymers comprising at least one repeating unit chosen from those of formulae (IIIa), (IIIb) and (IIIc), wherein at least one radical chosen from R1, R2, R3, and R4 of formula (IIIa), and R1 and R2 of formulae (IIIb) and (IIIc) is a carboxylic acid solubilizing group, in neutralized or non-neutralized form, optionally connected to the ring via a spacer, such as a spacer chosen from linear and branched C₁-C₂₀ alkyl radicals, and wherein the other radical(s) are hydrogen atoms.

The at least one conductive polymer can be present in the composition in an amount of at least 0.001% by weight, for instance, of at least 0.01% by weight, such as of at least 0.1% by weight and of at least 0.5% by weight, relative to the total weight of the composition. Moreover, the amount of the at least one conductive polymer present in the composition can be, for example, up to 50% by weight, such as up to 30% by weight, for instance up to 20% by weight and up to 10% by weight, relative to the total weight of the composition.

According to still another embodiment of the present disclosure, the at least one conductive polymer is present in the composition in an amount ranging from 0.1% to 50% by weight, for instance, from 0.1% and 30% by weight, such as from 0.5% and 10% by weight, relative to the total weight of the composition.

As disclosed herein, the composition according to the present disclosure comprises, in addition to the at least one conductive polymer, at least one fluorescent dye and/or at least one optical brightener, or a mixture thereof.

Fluorescent dyes are for example, compounds chosen from those that absorb light in the visible part of the spectrum and possibly in the ultraviolet region, and re-emit fluorescent light in the visible spectrum, of a longer wavelength than that of the absorbed light. For instance, the wavelength of the light re-emitted by the fluorescent dye may range from 500 nm to 650 nm.

In accordance with the present disclosure, the at least one fluorescent dye may be in a form that is soluble or insoluble in the medium of the composition, at room temperature, i.e., ranging from 15° C. to 25° C.

For example, the at least one fluorescent dye may be chosen from compounds that are soluble in the medium of the composition.

According to one embodiment of the present disclosure, for example, the solubility of the at least one fluorescent dye in the medium of the composition is at least 0.001 g/l, for instance at least 0.5 g/l, such as at least 1 g/l and at least 5 g/l, at a temperature ranging from 15° C. to 25° C.

Among the fluorescent dyes that are suitable for use according to the present disclosure, non-limiting mention may be made of those chosen from, for instance, naphthalimides; cationic and non-cationic coumarins; xanthenodiquinolizines; azaxanthenes; naphtholactams; azlactones; oxazines; thiazines; dioxazines, pyrenes, nitrobenzoxadiazoles, and mixtures thereof.

According to another embodiment of the present disclosure, the at least one fluorescent dye is chosen from those of formula: 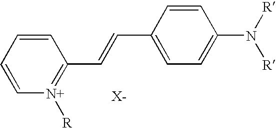
wherein:

R is chosen from linear and branched alkyl radicals comprising 1 to 22 carbon atoms, optionally substituted with at least one hydroxyl radical;

R′, which may be identical or different, are chosen from hydrogen atoms; and linear and branched alkyl radicals comprising 1 to 22 carbon atoms, such as 1 to 10 carbon atoms, optionally substituted with at least one hydroxyl radical;

X is chosen from organic and mineral anions.

For example, the radicals R′, which may be identical or different, may be chosen from hydrogen atoms and methyl radicals.

For further example, X³¹ may be an anion of mineral origin chosen from, for instance, halides, sulfates, bisulfates, nitrates, phosphates, hydrogen phosphates, dihydrogen phosphates, carbonates and bicarbonates. The anion X⁻ may also be of organic origin, such as those derived from salts of mono- or polycarboxylic acids, sulfonic acids and sulfuric acids, which are saturated or unsaturated, aromatic or non-aromatic, and optionally substituted with at least one hydroxyl or amino radical, or halogens.

In one embodiment, X⁻ is chosen from chloride, iodide, sulfate, methosulfate and ethosulfate.

Non-limiting examples of compounds of this type that may be mentioned include the compound having the formula: 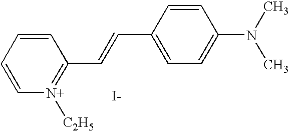
which is sold by the company Ubichem under the name Photosensitiving Dye NK-557. The compound comprising, in place of the alkyl group on the quaternized nitrogen atom, a methyl group, is also suitable for use.

Additional non-limiting examples of compounds that are also suitable for use include those of the formulae:

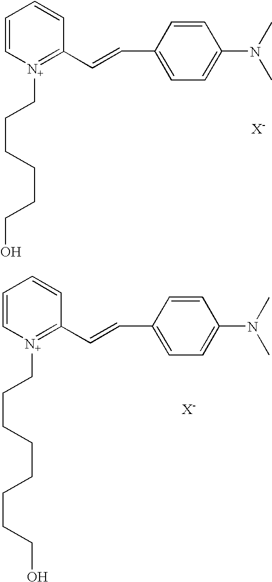

According to another embodiment of the present disclosure, the at least one fluorescent dye is chosen from the compounds of the formulae:

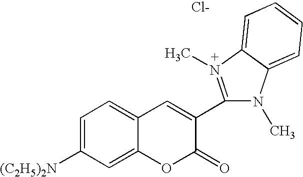

This compound corresponds to Brilliant Yellow B6GL sold by the company Sandoz; and

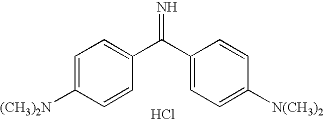

This compound corresponds to Basic Yellow 2, or Auramine O, sold by the companies Prolabo, Aldrich or Carlo Erba.

According to yet another embodiment of the present disclosure, the at least one fluorescent dye is present in an amount ranging from 0.01% to 20% by weight, for instance from 0.05% to 10% by weight, such as from 0.1% to 5% by weight, relative to the total weight of the composition.

The at least one optical brightener that may be present in the composition may be, for example, chosen from compounds that absorb light in the ultraviolet part of the spectrum, such as in the UVA region, at a wavelength ranging from 300 nm to 390 nm. These compounds re-emit a fluorescent light in the visible spectrum, at a wavelength ranging from 400 nm to 525 nm.

For example, optical brighteners that are soluble in the medium of the composition may be used. In one embodiment of the present disclosure, the at least one optical brightener is chosen from compounds that are soluble in the medium of the composition to at least 0.1 g/l for instance at least 0.5 g/l, at a temperature ranging from 15° C. to 25° C.

Among the optical brighteners that are suitable for use as disclosed herein, non-limiting mention may be made of stilbene derivatives, coumarin derivatives, oxazole and benzoxazole derivatives, and imidazole derivatives.

Further non-limiting examples include:
the stilbene derivative of naphthotriazole (Tinopal GS from Ciba), disodium 4,4′-distyrylbiphenyl sulfonate (CTFA name: disodium distyrylbiphenyl disulfonate; Tinopal CBS-X from Ciba: sodium 4,4′-bis[(4,6-dianilino-1,3,5-triazin-2-yl)amino]stilbene-2,2′-disulfonate), the cationic derivative of aminocoumarin (Tinopal SWN Conc. from Ciba), diethylaminomethylcoumarin, 4-methyl-7-diethylcoumarin, sodium 4,4′-bis[(4,6-dianilino-1,3,5-triazin-2-yl)amino]stilbene-2,2′-disulfonate (Tinopal SOP from Ciba), 4,4′-bis[(4-anilino-6-bis(2-hydroxyethyl)amino-1,3,5-triazin-2-yl)amino]stilbene-2,2′-disulfonic acid (Tinopal UNPA-GX from Ciba), 4,4′-bis[anilino-6-morpholine-1,3,5-triazin-2-yl)amino]-stilbene (Tinopal AMS-GX from Ciba), disodium 4,4′-bis[(4-anilino-6-(2-hydroxyethyl)methylamino-1,3,5-triazin-2-yl)amino]stilbene-2,2′-sulfonate (Tinopal 5BM-GX from Ciba),
2,5-thiophenediylbis(5-tert-butyl-1,3-benzoxazole) (Uvitex OB from Ciba),
the anionic derivative of diaminostilbene (dispersion in water, Leucophor BSB liquid from Clariant), and
optical brightener lakes (Covazur range from Wackherr).

The optical brightener may be present in an amount ranging from 0.01% to 20% by weight, such as from 0.05% and 10% by weight, for instance, from 0.1% and 5% by weight, relative to the total weight of the composition.

In another embodiment of the present disclosure, the composition does not comprise fluorescein as the at least one fluorescent compound.

The composition as disclosed herein may also comprise at least one non-fluorescent direct dye and/or at least one oxidation dye.

The non-fluorescent direct dyes may be chosen from nonionic, cationic and anionic direct dyes. For example, the direct dyes may be chosen from nitrobenzene dyes, azo, azomethine, methine, anthraquinone, naphthoquinone, benzoquinone, phenothiazine, indigoid, xanthene, phenanthridine, phthalocyanin and triarylmethane-based dyes, and also natural dyes, alone or as mixtures. If the dye composition comprises at least one non-fluorescent direct dye, it may be present in an amount ranging from 0.0005% to 12%, such as from 0.005% to 6% by weight, relative to the weight of the dye composition.

Oxidation dyes are compounds that are conventionally used in the field of dyeing and comprise at least one oxidation base optionally combined with at least one coupler.

Among the oxidation bases that are suitable for use as disclosed herein, non-limiting mention may be made of ortho- and para-phenylenediamines, double bases, for instance bis(phenyl)alkylenediamines, ortho- and para-aminophenols and heterocyclic bases, and also the acid addition salts thereof. If at least one oxidation base is used in the composition, it may be present in an amount ranging from 0.0005% to 12%, for instance from 0.005% to 8% by weight, relative to the weight of the dye composition.

Among the couplers that may be used as disclosed herein, non-limiting mention may be made of meta-aminophenols, meta-phenylenediamines, meta-diphenols, naphthols and heterocyclic couplers, and the acid addition salts thereof. When at least one coupler is used in the composition as disclosed herein, it may be present in an amount ranging from 0.0001% to 10% by weight, for instance from 0.005% to 5% by weight, relative to the weight of the dye composition.

The composition may also comprise nonionic, anionic, cationic, amphoteric or zwitterionic surfactants, and among these, non-limiting mention may be made of alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and also other nonionic surfactants of the hydroxypropyl ether type.

When the composition comprises at least one surfactant, it may be present in an amount less than 30% by weight, for instance ranging from 0.5% to 10% by weight, relative to the weight of the composition.

The cosmetically acceptable medium of the composition may comprise water, for example, or a mixture of water and at least one organic solvent that is acceptable in the field.

Among the organic solvents that may be used, non-limiting mention may be made, for example, of C₁-C₄ alcohols such as ethyl alcohol, isopropyl alcohol, aromatic alcohols such as benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers, for instance diethylene glycol monoethyl ether or monobutyl ether, or alternatively polyols, for instance glycerol. Polyethylene glycols and polypropylene glycols and mixtures of all these compounds may also be used as solvent.

The organic solvents, when present, may be present in an amount ranging from 1% to 40% by weight, for instance, from 5% to 30% by weight, relative to the total weight of the composition.

The dye composition may also comprise at least one conventional additive, known elsewhere in the dyeing of human keratin fibers, such as antioxidants, fragrances, dispersants, conditioners especially including cationic or amphoteric polymers, opacifiers, sequestering agents such as EDTA and etidronic acid, UV-screening agents, waxes, volatile or non-volatile, cyclic or linear or branched silicones, which are organomodified (such as by amine groups) or unmodified, preserving agents, ceramides, pseudoceramides, plant, mineral or synthetic oils, vitamins or provitamins, for instance panthenol, and nonionic, anionic, amphoteric or cationic associative polymers.

The composition may also comprise thickeners, for instance nonionic, anionic or amphoteric associative polymers, water-soluble thickener polymers of synthetic or natural origin, for instance polyvinylpyrrolidine, polyacrylic acid, polyacrylamide and polysaccharides of animal, plant or microbial origin.

If at least one thickener is present, it may be present in the composition in an amount ranging from 0.01% to 10% by weight, for instance, from 0.1% to 5% by weight, relative to the total weight of the composition.

The pH of the composition can generally range from 2 to 12, such as from 4 to 11. The pH may be adjusted to the desired value using acidifying or basifying agents.

Among the acidifying agents that may be used, non-limiting examples that may be mentioned include mineral or organic acids, for instance hydrochloric acid, orthophosphoric acid or sulfuric acid; organic acids such as sulfonic acids, carboxylic acids, for instance acetic acid, tartaric acid, citric acid and lactic acid.

Among the basifying agents that may be used, non-limiting mention may be made of those chosen from urea, alkali metal or alkaline-earth metal silicates and phosphates, ammonia precursor compounds, alkaline carbonates, alkanolamines such as monoethanolamine, diethanolamine and triethanolamine, and also derivatives thereof, sodium hydroxide, potassium hydroxide and the compounds of formula (A): 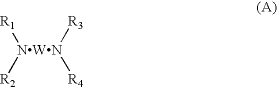
wherein W is a propylene residue optionally substituted with an entity chosen from hydroxyl radicals and C₁-C₆ alkyl radicals; R₁, R₂, R₃ and R₄, which may be identical or different, are chosen from hydrogen atoms and C₁-C₆ alkyl and C₁-C₆ hydroxyalkyl radicals.

According to one embodiment of the present disclosure, the composition comprises at least one oxidizing agent.

The oxidizing agent may be chosen, for example, from hydrogen peroxide, urea peroxide, alkali metal bromates, persalts such as perborates and persulfates, and enzymes such as the peroxidases and two- or four-electron oxidoreductases. In one embodiment of the present disclosure, the at least one oxidizing agent is chosen from hydrogen peroxide and enzymes.

The amount of oxidizing agent, when present, may range from 0.001% to 10% by weight, relative to the weight of the composition.

The composition according to the present disclosure may be in various forms, such as lotions, shampoos, creams, gels or pastes, or in any other suitable cosmetic form.

The processes according to the present disclosure are applicable to keratin fibers, for instance human keratin fibers, for example such as the hair.

In accordance with one embodiment according to the present disclosure, the process comprises:
applying to wet or dry keratin fibers the composition as disclosed herein, and leaving the composition on the fibers for a time that is sufficient to obtain the desired effect;
optionally rinsing the fibers,
optionally, washing and rinsing the fibers,
drying the fibers or leaving them to dry.

The above process may be performed, for example, in the case where an oxidizing agent is present, or in the case where an oxidation dye is present (i.e. at least one oxidation base optionally combined with at least one coupler). When at least one oxidizing agent is desired, the composition according to the present disclosure and the at least one oxidizing agent may be applied sequentially in any order, or even simultaneously. If simultaneous application is chosen, the composition and the at least one oxidizing agent can be for example, mixed together just before application. Moreover, it should be noted that, In the case of the above embodiment, the leave-in time of the composition may range from 5 to 60 minutes, such as from 5 to 40 minutes.

According to another embodiment of the present disclosure, a process for treating human keratin fibers comprises:
applying the composition as disclosed herein to wet or dry keratin fibers,
drying the fibers or leaving them to dry. The temperature at which the composition is applied to the fibers may range from 15° C. to 80° C., for instance from 15° C. to 40° C.

In accordance with another embodiment, it is possible to separately store the composition according to the present disclosure, on the one hand, and an oxidizing composition, on the other hand. The oxidizing composition comprises, for example, at least one oxidizing agent in a medium that is suitable for dyeing. The oxidizing composition may obviously comprise other ingredients, for instance nonionic, anionic, cationic or amphoteric surfactants, and also any other additive conventionally used in the field. Reference may thus be made to the additives mentioned in the context of the description of the composition according to the present disclosure above.

The composition as disclosed herein and the oxidizing composition are then mixed together at the time of use, after which this mixture is applied to the keratin fibers; the other abovementioned steps of the processes above then being performed.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The example that follows illustrates the present disclosure without limiting the scope thereof.

### EXAMPLE

### Synthesis of poly(thiophene-3-acetic acid)

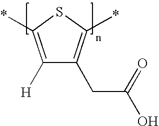 Procedure

Preparation of the polymer: poly(ethyl thiophene-3-acetate)

25 ml of dry chloroform were introduced into a Schlenk tube under argon, the system is degassed and the following reagents were then introduced: 2.5 g of ethyl thiophene-3-acetate (14.7 mmol) 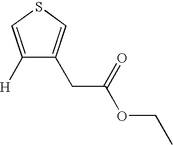
and 1 g of FeCl₃ (6.15 mmol).

The mixture was stirred for 24 hours under argon at 50° C.

The poly(ethyl thiophene-3-acetate) polymer was then precipitated in heptane. The polymer was then dissolved in a tetrahydrofuran solution.

Infrared characterization: C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the polymer: poly(ethyl thiophene-3-acetate) to form poly(thiophene-3-acetic acid)

The polymer obtained above was then hydrolyzed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared characterization of the polymer: C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation comprising the polymer and process for using it:

| | |
|---|---|
| Poly(thiophene-3-acetic acid) | 5 g |
| Aminomethylpropanol qs | pH 7 |
| 2-[2-(4-dimethylamino)phenyl- | |
| ethenyl]-1-ethyl-N-pyridinium | |
| iodide (from the company Ubichem) | 0.15 g |
| Ethyl alcohol | 20 g |
| Water qs | 100 g |

The formula was applied to dark hair.

After a standing time of 20 minutes, drying (drying in air) was performed.

1. A composition comprising, in a cosmetically acceptable medium:
at least one fluorescent dye and/or at least one optical brightener, and
at least one conductive polymer.

2. A composition according to claim 1, wherein the at least one conductive polymer comprises at least one repeating unit of the following formulae:
anilines of formula (I) below: 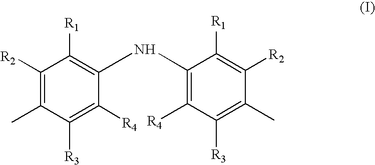
pyrroles of formulae (IIa) and (IIb): 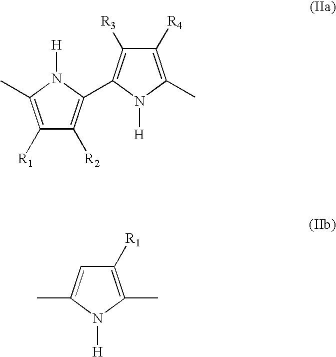
thiophenes and bisthiophenes of formulae (IIIa), (IIIb) and (IIIc): 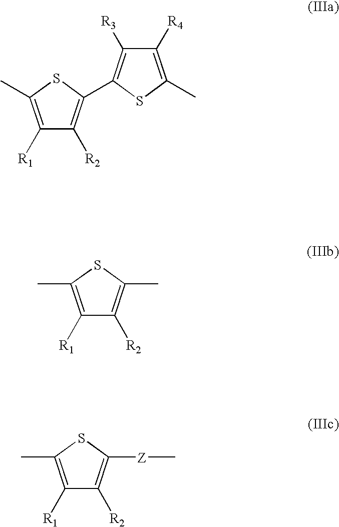
furans of formula (IV): 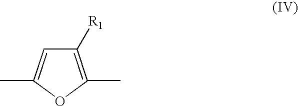
para-phenylene sulfides of formula (V): 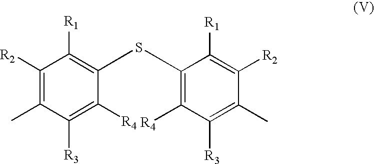
para-phenylenevinylenes of formula (VI): 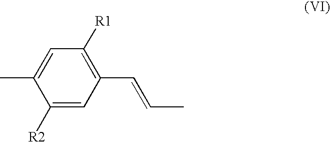
indoles of formula (VII): 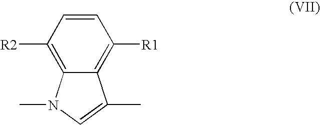
aromatic amides of formulae (VIIIa), (VIIIb), (VIIIc) and (VIIId): 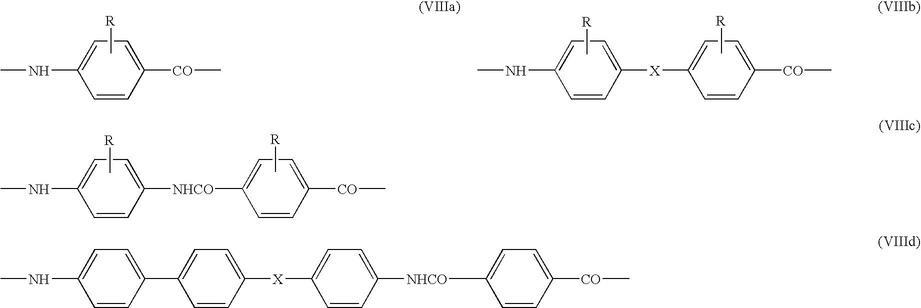
aromatic hydrazides of formulae (IXa), (IXb) and (IXc): 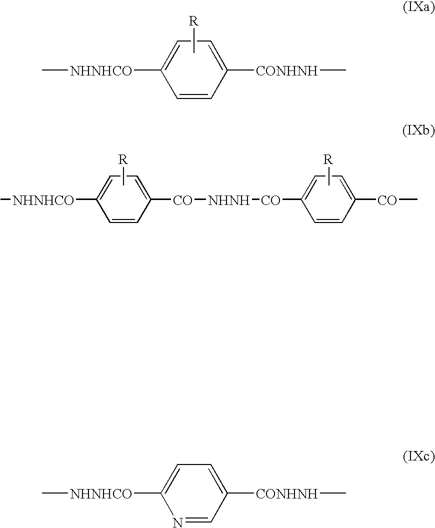
aromatic azomethines of formulae (Xa), (Xb) and (Xc): 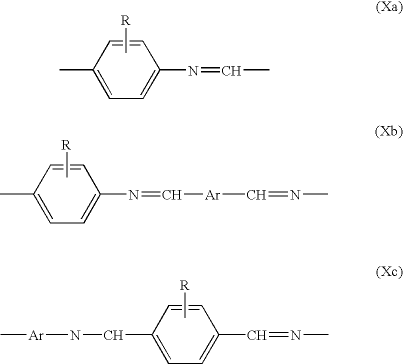
aromatic esters of formulae (XIa), (XIb) and (XIc): 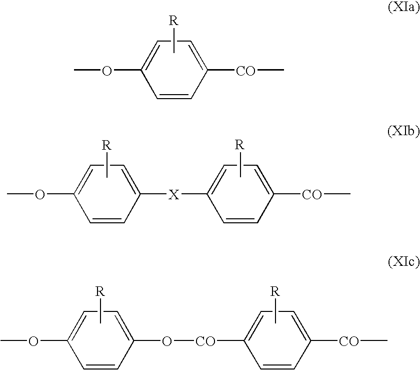
wherein in formulae (I) to (XI):
the radicals R, R1, R2, R3, and R4, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups, and solubilizing groups comprising a spacer group that bonds to the ring;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

3. The composition according to claim 2, wherein the solubilizing groups are chosen from
—COOH and —COO-M⁺ radicals, wherein M is chosen from alkali metals, alkaline-earth metals, organic amines, alkanolamines and amino acids,
—SO₃H and —SO₃⁻M⁺ radicals, wherein M is chosen from alkali metals, alkaline-earth metals, organic amines, alkanolamines and amino acids,
primary, secondary and tertiary amine radicals,
quatemary ammonium radicals,
hydroxyl radicals, and
poly((C₂-C₃)alkylene oxide) radicals.

4. The composition according to claim 3, wherein the quaternary ammonium radicals are —N(R′)₃⁺Z⁻ radicals wherein Z is chosen from Br and Cl atoms, and (C₁-C₄)alkyl-OSO₃ radicals, and wherein R′, which may be identical or different, are chosen from linear and branched C₁ to C₂₀ alkyl radicals, or two R′ radicals can form a heterocycle with the nitrogen.

5. The composition according to claim 3, wherein the solubilizing groups are connected to the ring via a spacer group.

6. The composition according to claim 5, wherein the spacer group is chosen from —R″—, —OR″—, —OCOR″— and —COOR″— radicals, wherein R″ is chosen from linear and branched C₁-C₂₀ alkyl radicals optionally comprising at least one hetero atom.

7. The composition according to claim 2, wherein the radicals R, R1, R2, R3, and R4, which may be identical or different, are chosen from hydrogen atoms, and R′, —OR′, —OCOR′ and —COOR′ radicals, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃ with x being an average number ranging from 0 to 200.

8. The composition according to claim 2, wherein at least one radical chosen from R, R1, R2, R3, and R4 of the at least one conductive polymer is a solubilizing group.

9. The composition according to claim 2, wherein the at least one conductive polymer comprises at least one solubilizing group per repeating unit.

10. The composition according to claim 3, wherein the solubilizing groups are chosen from carboxylic acid radicals; sulfonic acid radicals; tertiary amine radicals; quaternary ammonium radicals; and also the salts thereof; wherein the solubulizing groups are optionally connected to the ring via a spacer group; and wherein the carboxylic acid and sulfonic acid radicals may optionally be neutralized.

11. The composition according to claim 10, wherein the quaternary ammonium radicals are —N(R′)₃⁺Z⁻ radicals, wherein Z is chosen from Br and Cl atoms, and (C₁-C₄)alkyl-OSO₃ radicals, and wherein R′, which may be identical or different, is chosen from linear and branched C₁-C₂₀ alkyl radicals.

12. The composition according to claim 10, wherein the solubilizing groups are connected to the ring via a spacer chosen from C₁-C₂₀ alkyl radicals.

13. The composition according to claim 2, wherein the at least one conductive polymer is chosen from polymers comprising at least one repeating unit of formula (IIIa), (IIIb) or (IIIc), wherein at least one radical chosen from R1, R2, R3, and R4 of formula (IIIa) and R1 and R2 of formulae (IIIb) and (IIIc) is a a solubilizing group of carboxylic acid type, in neutralized or non-neutralized form, optionally connected to the ring via a spacer group, and wherein the other radicals are hydrogen atoms.

14. The composition according to claim 13, wherein the spacer group is chosen from linear and branched C₁-C₂₀ alkyl radicals.

15. The composition according to claim 1, wherein the at least one conductive polymer is present in an amount of at least 0.001% by weight, relative to the total weight of the composition.

16. The composition according to claim 1, wherein the at least one conductive polymer is present in an amount of up to 50% by weight, relative to the total weight of the composition.

17. The composition according to claim 1, wherein the at least one conductive polymer is present in an amount ranging from 0.1% to 50% by weight, relative to the total weight of the composition.

18. The composition according to claim 1, wherein the at least one fluorescent dye is chosen from compounds that absorb light in the visible part of the spectrum and optionally in the ultraviolet region, and re-emit a fluorescent light in the visible part of the spectrum, of a longer wavelength than that of the absorbed light.

19. The composition according to claim 18, wherein the at least one fluorescent dye re-emits a light with a wavelength ranging from 500 nm to 650 nm.

20. The composition according to claim 1, wherein the at least one fluorescent dye is chosen from compounds that are soluble in the medium of the composition.

21. The composition according to claim 1, wherein the at least one fluorescent dye is chosen from naphthalimides; cationic and non-cationic coumarins; xanthenodiquinolizines; azaxanthenes; naphtholactams; azlactones; oxazines; thiazines; dioxazines, pyrenes, nitrobenzoxadiazoles, and mixtures thereof.

22. The composition according to claim 18, wherein the at least one fluorescent dye is chosen from those of formula: 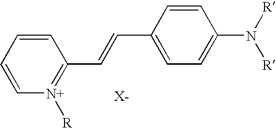
wherein:
R is chosen from linear and branched alkyl radicals comprising 1 to 22 carbon atoms, optionally substituted with at least one hydroxyl radical;
R′, which may be identical or different, are chosen from hydrogen atoms; and linear and branched alkyl radicals comprising 1 to 22 carbon atoms, optionally substituted with at least one hydroxyl radical; and
X is chosen from organic and mineral anions.

23. The composition according to claim 22, wherein R′, which may be identical or different, are chosen from hydrogen atoms and linear and branched alkyl radicals comprising 1 to 10 carbon atoms, optionally substituted with at least one hydroxyl radical.

24. The composition according to claim 18, wherein the at least one fluorescent dye is chosen from the following formulae:

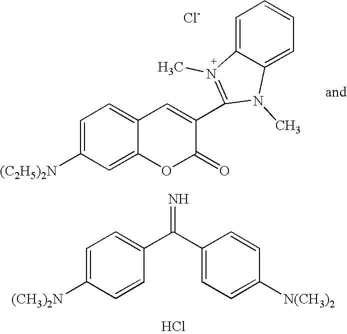

25. The composition according to claim 18, wherein the at least one fluorescent dye is present in an amount ranging from 0.01% to 20% by weight, relative to the total weight of the composition.

26. The composition according to claim 25, wherein the at least one fluorescent dye is present in an amount ranging from 0.05% to 10% by weight, relative to the total weight of the composition.

27. The composition according to claim 26, wherein the at least one fluorescent dye is present in an amount ranging from 0.1% to 5% by weight, relative to the total weight of the composition.

28. The composition according to claim 1, wherein the at least one optical brightener is chosen from compounds that are soluble in the medium of the composition.

29. The composition according to claim 28, wherein the at least one optical brightener is chosen from stilbene derivatives, coumarin derivatives, oxazole and benzoxazole derivatives and imidazole derivatives.

30. The composition according to claim 1, wherein the at least one optical brightener is present in an amount ranging from 0.01% to 20% by weight, relative to the total weight of the composition.

31. The composition according to claim 30, wherein the at least one optical brightener is present in an amount ranging from 0.05% to 10% by weight, relative to the total weight of the composition.

32. The composition according to claim 31, wherein the at least one optical brightener is present in an amount ranging from 0.1% to 5% by weight, relative to the total weight of the composition.

33. The composition according to claim 1, wherein the cosmetically acceptable medium comprises water or a mixture of water and at least one organic solvent.

34. The composition according to claim 33, wherein the at least one solvent is chosen from alcohols, glycols, glycol ethers, polyols, polyethylene glycols, polypropylene glycol, and mixtures thereof.

35. The composition according to claim 1, further comprising at least one surfactant chosen from nonionic, anionic, cationic, amphoteric and zwitterionic surfactant.

36. The composition according to claim 35, wherein the at least one surfactant is present in an amount less than 30% by weight, relative to the weight of the composition.

37. The composition according to claim 36, wherein the at least one surfactant is present in an amount ranging from 0.5% to 10% by weight, relative to the weight of the composition.

38. The composition according to claim 1, further comprising at least one non-fluorescent direct dye chosen from nonionic, cationic and anionic non-fluorescent direct dyes.

39. The composition according to claim 38, wherein the at least one non-fluorescent direct dye is chosen from nitrobenzene dyes, azo, azomethine, methine, anthraquinone, naphthoquinone, benzoquinone, phenothiazine, indigoid, xanthene, phenanthridine, phthalocyanin, triarylmethane-based dyes, natural dyes, and mixtures thereof.

40. The composition according to claim 38, wherein the at least one non-fluorescent direct dye is present in an amount ranging from 0.0005% to 12% by weight, relative to the total weight of the composition.

41. The composition according to claim 1, further comprising at least one oxidation base chosen from para-phenylenediamines, double bases, para-aminophenols, ortho-aminophenols, heterocyclic bases, and the acid addition salts thereof.

42. The composition according to claim 41, wherein the at least one oxidation base is present in an amount ranging from 0.0005% to 12% by weight, relative to the total weight of the composition.

43. The composition according to claim 1, further comprising at least one coupler chosen from meta-phenylenediamines, meta-aminophenols, meta-diphenols, heterocyclic couplers, and the acid addition salts thereof.

44. The composition according to claim 43, wherein the at least one coupler is present in an amount ranging from 0.0001% to 10% by weight, relative to the total weight of the dye composition.

45. The composition according to claim 1, further comprising at least one oxidizing agent.

46. A process for treating keratin fibers, comprising:
applying to wet or dry keratin fibers a composition comprising, in a cosmetically acceptable medium:
at least one fluorescent dye and/or at least one optical brightener, and
at least one conductive polymer
and leaving the composition on the fibers for a period of time that is sufficient to obtain a desired coloration effect;
optionally rinsing the fibers,
optionally washing and rinsing the fibers, and
drying the fibers or leaving them to dry.

47. The process according to claim 46, wherein the at least one conductive polymer comprises at least one repeating unit of the following formulae:
anilines of formula (I) below: 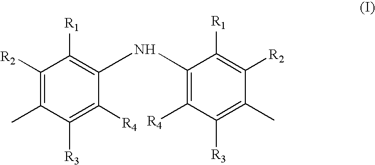
pyrroles of formulae (IIa) and (IIb): 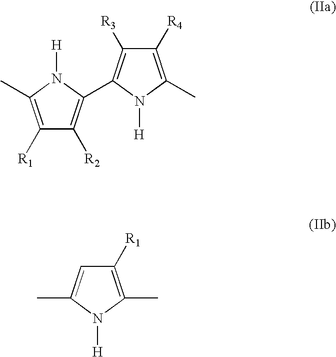
thiophenes and bisthiophenes of formulae (IIIa), (IIIb) and (IIIc): 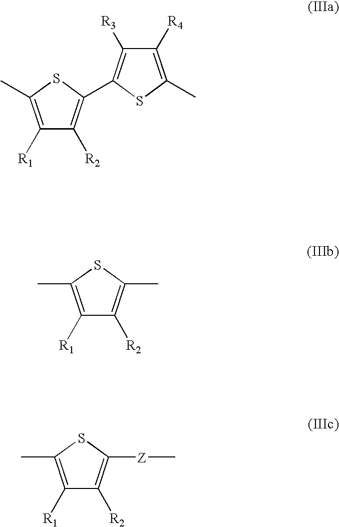
furans of formula (IV): 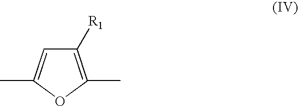
para-phenylene sulfides of formula (V): 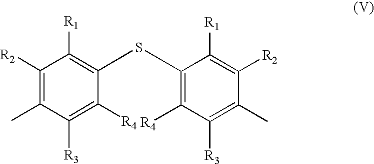
para-phenylenevinylenes of formula (VI): 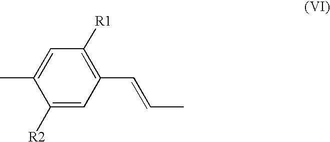
indoles of formula (VII): 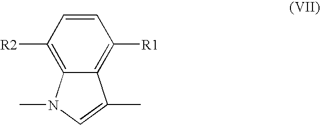
aromatic amides of formulae (VIIIa), (VIIIb), (VIIIc) and (VIIId): 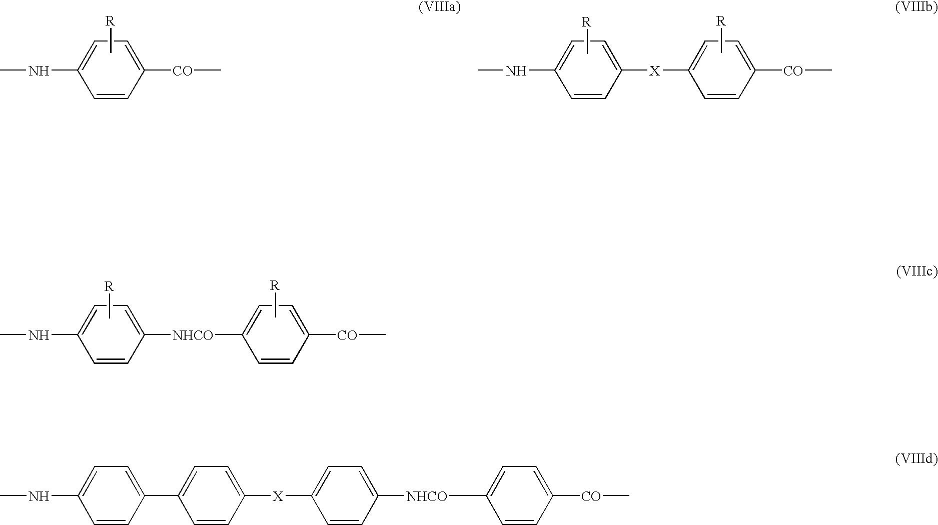
aromatic hydrazides of formulae (IXa), (IXb) and (IXc): 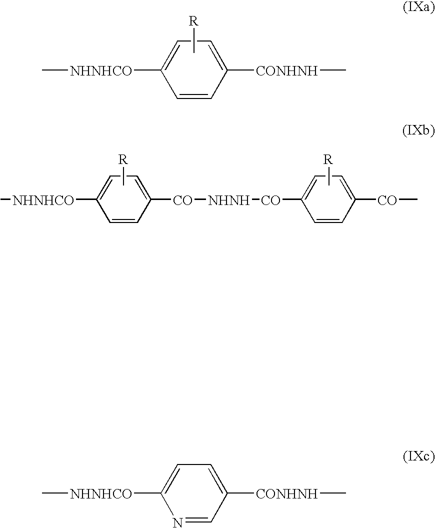
aromatic azomethines of formulae (Xa), (Xb) and (Xc): 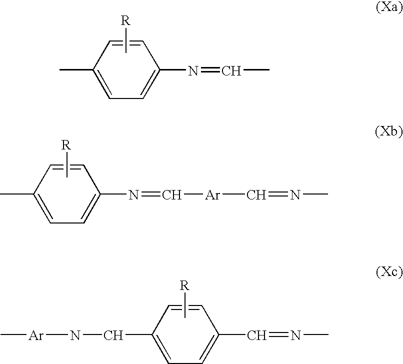
aromatic esters of formulae (XIa), (XIb) and (XIc): 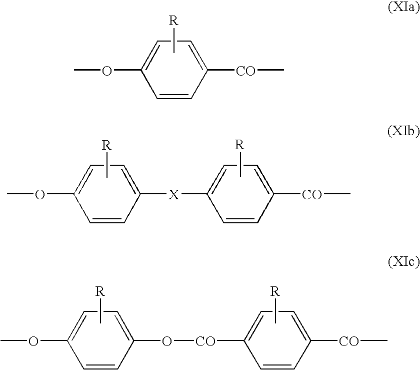
wherein in formulae (I) to (XI):
the radicals R, R1, R2, R3, and R4, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups, and solubilizing groups comprising a spacer group that bonds to the ring;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

48. The process according to claim 47, wherein the solubilizing groups are chosen from
—COOH and —COO-M⁺ radicals, wherein M is chosen from alkali metals, alkaline-earth metals, organic amines, alkanolamines and amino acids,
—SO₃H and —SO₃⁻M⁺ radicals, wherein M is chosen from alkali metals, alkaline-earth metals, organic amines, alkanolamines and amino acids,
primary, secondary and tertiary amine radicals,
quaternary ammonium radicals,
hydroxyl radicals, and
poly((C₂-C₃)alkylene oxide) radicals.

49. The process according to claim 46, wherein the at least one fluorescent dye is chosen from compounds that absorb light in the visible part of the spectrum and optionally in the ultraviolet region, and re-emit a fluorescent light in the visible part of the spectrum, of a longer wavelength than that of the absorbed light.

50. The process according to claim 46, wherein the at least one optical brightener is chosen from compounds that are soluble in the medium of the composition.

51. A process for treating keratin fibers, comprising:
applying to wet or dry keratin fibers a composition comprising, in a cosmetically acceptable medium, at least one fluorescent dye and/or at least one optical brightener, and- at least one conductive polymer; and
drying the fibers or leaving the fibers to dry.

52. A method for cosmetically treating keratin fibers, comprising, applying to the keratin fibers, in a cosmetically acceptable medium, at least one conductive polymer and at least one fluorescent dye and/or at least one optical brightener, wherein the at least one conductive polymer and the at least one fluorescent dye and/or at least one optical brightener are present in an amount sufficient to impart to the fibers an optical effect.

53. The method according to claim 52, wherein the optical effect is a sheen effect.

54. The composition according to claim 1, wherein the at least one conductive polymer has a conductivity ranging from 10⁻⁵ to 5×10⁻⁵ siemens/cm.

55. A kit comprising:
at least one first compartment comprising a composition comprising, in a cosmetically acceptable medium:
at least one fluorescent dye and/or at least one optical brightener, and
at least one conductive polymer, and optionally
at least one second compartment comprising at least one oxidizing composition comprising at least one oxidizing agent.
